(19) United States
(12) Patent Application Publication
(10) Pub. No.: US 2008/0200536 A1
(43) Pub. Date: Aug. 21, 2008

(54) Pharmaceutical Formulation with High Stability and Dissolution and Manufacturing Process

(75) Inventors: Joo Myung Moon, Gyeonggi-do (KR); Hyun-Ah Lee, Gyeonggi-do (KR)

Correspondence Address: HUGHES LAW FIRM, PLLC, PACIFIC MERIDIAN PLAZA, SUITE 302, 4164 MERIDIAN STREET, BELLINGHAM, WA, 98226-5583, US

(73) Assignee: BORAM PHARM.CO., LTD, GYEONGGI-DO (KR)

(21) Appl. No.: 12/063,090

(22) PCT Filed: Jun. 26, 2006
(86) PCT No.: PCT/KR2006/002452

§ 371 Date: Feb. 6, 2008

(30) Foreign Application Priority Data
Aug. 17, 2005 (KR) 10-2005-0075266

(51) Int. Cl. A61K 31/365

(52) U.S. Cl. 514/449

(57) ABSTRACT

Disclosed herein are a pharmaceutical formulation with high stability and dissolution, and a method for preparing the pharmaceutical formulation. The pharmaceutical formulation comprises a pharmacologically active substance, a solvent, a solubilizer, a surfactant, an antioxidant, and an adsorbent. According to the pharmaceutical formulation and the method, the pharmacologically active substance is mixed with the solvent, the solubilizer agent and the surfactant for improving the solubility of the pharmacologically active substance to obtain an amorphous liquid or semi-solid state, the antioxidant is melted together with the mixture to solve poor chemical stability of the pharmacologically active substance in an amorphous or liquid state, and the adsorbent is strongly adsorbed to the molten mixture so as to be transformed into a powder form so that the resulting molecules are reconstituted into very tiny crystal forms within the adsorbent to ensure chemical stability.

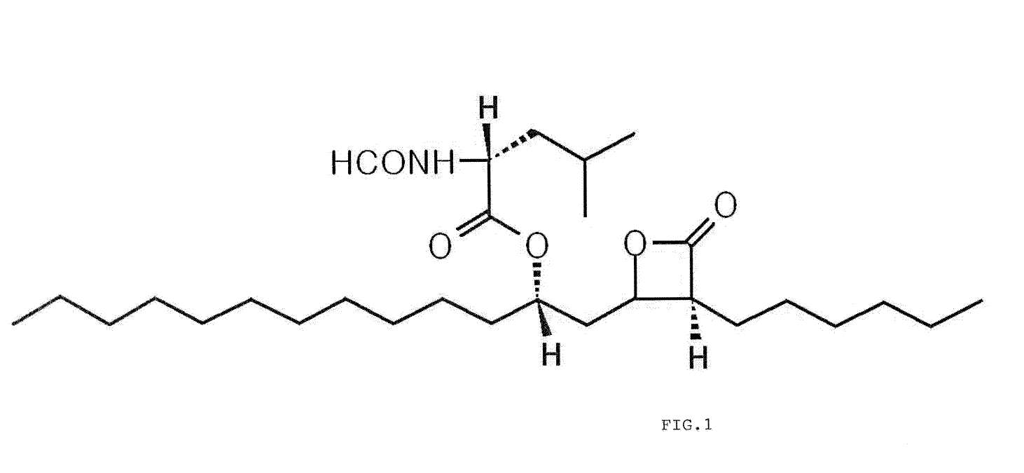

### TECHNICAL FIELD

The present invention relates to a pharmaceutical formulation with high stability and dissolution, and a method for preparing the pharmaceutical formulation. More specifically, the present invention relates to a pharmaceutical formulation comprising a pharmacologically active substance, a solvent, a solubilizer, a surfactant, an antioxidant and an adsorbent wherein the pharmacologically active substance is mixed with the solvent, the solubilizer agent and the surfactant for improving the solubility of the pharmacologically active substance to obtain an amorphous liquid or semi-solid state, the antioxidant is melted together with the mixture to solve poor chemical stability of the pharmacologically active substance in an amorphous or liquid state, and the adsorbent is strongly adsorbed to the molten mixture so as to be transformed into a powder form so that the resulting molecules are reconstituted into very tiny crystal forms within the adsorbent to ensure chemical stability, the characteristic porous or cellulosic structure of the adsorbent blocks and protects the pharmacologically active substance from factors, e.g., air and moisture, causing chemical instability of the pharmacologically active substance, and the final pharmaceutical formulation is stabilized within a pH of 4.5 to 5.5.

### BACKGROUND ART

In most cases, the solubility of drugs depends on the crystal forms of the drug ingredients. The fact is generally known that high crystallinity of drugs leads to poor solubility and low bioavailability of the drugs. Accordingly, disruption of the crystallinity of poorly soluble drugs and transformation of the crystallinity into an amorphous state are of the greatest importance for the improvement of the bioavailability of the drugs.

In this connection, various methods have been known or suggested, for example:

1) a method for preparing a mixture of a pharmacologically active substance and a dispersant by simultaneously dissolving the pharmacologically active substance and the dispersant in an organic solvent to obtain a mixed solution and injecting the solution at a high speed to rapidly evaporate the organic solvent, thereby preventing the recrystallization of the pharmacologically active substance;

2) a method for preparing an amorphous copolymer by melting a pharmacologically active substance having a low melting point together with a polymer compound having a melting point similar to that of the pharmacologically active substance, and rapidly cooling the molten mixture;

3) a method for preventing recrystallization of a pharmacologically active substance having a low molecular weight by dissolving the pharmacologically active substance in a solvent, and capturing the molecules of the pharmacologically active substance within beta-cyclodextrin cavities;

4) a method for preparing a liquid formulation or a soft capsule using a mixed solution of a pharmacologically active substance, a solubilizer and a surfactant;

5) a method for preparing a liquid or powder formulation of a pharmacologically active substance using lecithin liposomes, taking advantage of the physicochemical properties of lecithin to form a hydrophilic and lipophilic sphere layer;

6) a method for preparing a microemulsion, such as W/O, O/W, O/W/O a W/O/W emulsion, of a pharmacologically active substance; and

7) a method for preparing a mixture of a polymer compound and a crystalline pharmacologically active substance at an optimum temperature in a nanopowder form while stirring at a high speed to induce a solid diffusion, as taught in Korean Patent Application No. 10-2004-0044474.

The crystal forms of some drugs, particularly low-melting point drugs, play an important role in stabilizing the drugs. The amorphous forms of unstable compounds under general storage conditions may promote denaturation of the unstable compounds.

Accordingly, pharmaceutical formulations are required to have a crystal form in order to solve the problems arising from their low melting point and possible degradation under storage conditions, and conversely, they must be able to solve the problem of poor solubility resulting from their crystallinity. There is thus a need to develop a pharmaceutical formulation satisfying the requirements.

For example, orlistat(tetrahydrolipstatin) as a lipase inhibitor or its structurally related compounds are molecules that may be degraded during storage by different mechanisms. It is well known that the degradation rate of active compounds largely depends on the physicochemical states of the active compound.

Lipase inhibitors or their structurally related compounds keep their crystal forms to ensure stability during storage, but they accompany solubility difficulties resulting from their crystallinity. Accordingly, both stability and solubility have to be considered in drugs for oral administration. Factors damaging the chemical stability of drugs under general storage conditions are oxidation and reduction reactions. Therefore, it is necessary to design pharmaceutical formulations that are stable against air and moisture. The invention of International Patent Application No. PCT/EP2002/005958 is based on the findings that the eutectic temperature of a mixture of orlistat, a fatty acid or fatty acid salt and water is below body temperature and that a dry powder of the mixture is present in a powder form under storage conditions. According to this invention, the degradation of orlistat is retarded by previously providing a fatty acid ester, which is a degradation product of orlistat, to assist in maintaining the equilibrium of the chemical degradation. However, since the final state of the composition is amorphous as clearly stated in the patent publication, the chemical stability of the composition is incompletely ensured, the preparation involves complicated steps, and the stability is not continuously guaranteed.

International Patent Application No. PCT/EP2001/06834 describes a porous formulation which is expanded in solutions and dispersions. However, the disadvantage of the formulation is that the preparation procedure is very complicated.

### DISCLOSURE OF INVENTION

### Technical Problem

Therefore, the present invention has been made in view of the above problems, and it is an object of the present invention to provide a pharmaceutical formulation that has a crystal form to solve the problems arising from its low melting point and possible degradation under storage conditions, and conversely, can solve the problem of poor solubility resulting from its crystallinity.

### Technical Solution

In accordance with an aspect of the present invention for achieving the above object, there is provided a pharmaceutical formulation comprising a pharmacologically active substance, at least one solvent, at least one solubilizer, at least one surfactant, at least one antioxidant, at least one antioxidative synergist, and an adsorbent wherein the pharmacologically active substance is melted together with the solvent, the solubilizer agent and the surfactant, the antioxidant and the antioxidative synergist are added to ensure chemical stability, the adsorbent is adsorbed to the molten mixture to improve possible chemical instability of the pharmacologically active substance in a liquid state and induce the state of the mixture to a powder form, and the adsorbed mixture is uniformly dispersed so that the active substance is very finely recrystallized within the adsorbent due to a very strong adsorption surface tension.

### BEST MODE FOR CARRYING OUT THE INVENTION

The pharmacologically active substance is a substance that is poorly soluble, is unstable under storage conditions, resulting in degradation, and may be rapidly degraded in an amorphous or liquid state. The pharmacologically active substance is preferably a lipase inhibitor, and more preferably orlistat (tetrahydrolipstatin) or its analogue, for example, 2-oxy-4H-3,1-benzoxazin-4-one.

Orlistat is a lipase inhibitor represented by Formula 1 below:

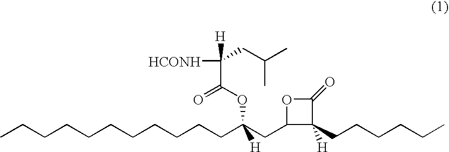

The term “lipase inhibitor” refers to a compound which is capable of inhibiting the action of lipase in the stomach and pancreas.

Orlistat is a drug having a melting point as low as 43° C., and is commercially available in a powder form. The dissolution rate of orlistat undergoing no denaturation under good storage conditions is about 60%. This low dissolution rate of orlistat does not meet the required level of bioavailability. In addition, when raw materials of orlistat are exposed to high temperature during transport, the powder particles of orlistat rapidly aggregate. Thereafter, the aggregates remain even when being cooled, causing damage to the dissolution of orlistat. As a result, the dissolution rate of orlistat is sharply reduced to 40% or lower.

The solubilizer is a pharmaceutically acceptable solvent for the purpose of increasing the bioavailability of the pharmacologically active substance. Examples of suitable solubilizers include solvents, such as almond oil, castor oil, corn oil, cotton seed oil, ethyl oleate, glycerin, glyceryl monostearate, olive oil, peanut oil, polyethylene glycol, propylene glycol and soy bean oil. Included also are solubilizers whose one functional group is bonded to the hydrophobic pharmacologically active substance and whose hydrophilic groups are not bonded to the pharmacologically active substance, after which the solubilizers are rapidly dissolved in water through the hydrophilic groups when in contact with water, to solubilize the poorly soluble active substance, and examples thereof include arabic gum, cetostearyl alcohol, cholesterol, diethanolamine, ethyl oleate, ethylene glycol palmitostearate, glycerin, glyceryl monostearate, hydroxypropyl cellulose, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, and triethanolmine. These solubilizers may be used alone or as a mixture thereof. The solubilizers are preferably present in a liquid state at room temperature. More preferred are polyethylene glycol and polyoxyethylene castor oil glycoside.

The surfactant serves to control the surface tension of lipophilic materials to increase the solubility of the lipophilic materials in water, and is also involved in the dispersion of the liquid-phase pharmacologically active substance. Exemplary surfactants include sodium docusate, glyceryl monooleate, polyethylene alkyl ether, polyoxyethylene sorbitan fatty acid ester (polysorbate=Tween), sodium lauryl sulfate, sorbic acid, and sorbitan fatty acid ester. The surfactant is preferably provided in an oily state, and is more preferably polysorbate. An auxiliary surfactant powder may also be used. As a preferred auxiliary surfactant, sodium lauryl sulfate is used.

The antioxidant plays a fundamental role in preventing the oxidation of the pharmacologically active substance to ensure the storage stability of the drug. In addition, antioxidants are known to prevent recrystallization and reaggregation of drugs in gastric acid after oral ingestion (see, Korean Patent Application No. 10-2004-0044475).

Examples of such antioxidants are tocopherol, ascorbic acid and its glycosides, butylated hydroxyanisole, citric acid, edetic acid, fumaric acid, malic acid, monothioglycerin, phosphoric acid, potassium metabisulfite, propionic acid, propyl gallate, and tartaric acid. The antioxidant preferably exists in a liquid state at room temperature, and is more preferably tocopherol related materials that are acceptable in pharmaceutical formulations.

The antioxidative synergist refers to a material that further enhances the antioxidizing power of the antioxidant. For example, when tocopherol is used as an antioxidant, citric acid may be added as an antioxidative synergist. In most cases, two or more antioxidants are used to create synergistic effects. Accordingly, the use of at least one antioxidant and at least one antioxidative synergist is included within the scope of the present invention.

Dispersants and adsorbents are largely distinguished in terms of their functions. That is, adsorbents function to disperse other materials by means of absorbing them, whereas dispersants function to uniformly disperse other materials within a matrix rather than to adsorb the materials. The adsorbent used in the present invention has a porous structure, and specifically refers to a material that is present in a colloidal amorphous form or a porous polymeric material. Examples of such adsorbents include: porous mineral materials, such as silicon dioxide, kaolin and magnesium aluminum silicate; polymers that fundamentally adsorb low molecular-weight materials within their structure, such as cyclodextrin and its derivatives, alginic acid and propylene glycol alginate; gums, such as arabic gum and xanthan gum; celluloses, such as cellulose powder, microcrystalline cellulose, ethyl cellulose, methyl cellulose, calcium carboxymethyl cellulose, sodium carboxymethyl cellulose, hydroxyethyl cellulose, hydroxymethyl cellulose, hydroxypropyl cellulose and hydroxypropylmethyl cellulose; polymers having a major function to disperse other materials, such as poloxamer, povidone and its derivatives, sodium starch glycolate and carbomer. In addition to these dispersants, dextrin, gelatin, medium-chain triglyceride, tragacanth, and the like, are good adsorbents and dispersants. These adsorbents may be used alone to perform adsorption/dispersion functions, but mixtures of two or more adsorbents are preferably used to create synergistic effects. Preferred is a mixture of a porous colloidal adsorbent and a cellulose type adsorbent. Further, a blend with a polymeric adsorbent is very useful. Colloidal silicon dioxide and microcrystalline cellulose are more preferably used as adsorbents, and polyvinyl pyrrolidone and sodium starch glycolate are more preferably used as dispersants.

Crystallinity required in maintaining good chemical stability of orlistat and its related materials is disadvantageous in terms of bioavailability. However, trans-formation of a crystal form into an amorphous form to increase bioavailability results in damage to chemical stability. An important point of the present invention is to provide solutions to satisfy the contradictory requirements. To this end, the present invention suggests the following solutions: reduction of excessive crystallinity, use of a solubilized composition, improvement of antioxidative ability, use of porous and cellulose type adsorbents to solve instability problems, e.g., hydrolysis, and determination of a pH value suitable to achieve maximum chemical stability.

A method for preparing the pharmaceutical formulation according to the present invention comprises the steps of:

mixing 0.01-20 parts by weight of the solvent, 0.01-20 parts by weight of the solubilizer, 0.01-10 parts by weight of the surfactant and 0.01-2 parts by weight of the antioxidant with heating to 40-60° C.;

mixing the mixture with 1 part by weight of the pharmacologically active substance;

adsorbing the mixture obtained in the previous step to 0.1-20 parts by weight of the adsorbent;

mixing the mixture obtained in the previous step with a pharmaceutically acceptable excipient suitable for molding; and

molding the mixture obtained in the previous step into a tablet followed by coating or capsule.

In the step of mixing with the pharmacologically active substance, solubilization is carried out as rapidly as possible to ensure the stability of the pharmacologically active substance. In the step of adsorbing to the adsorbent, a mixture of the adsorbent and a dispersant powder is fed into a vessel in which high-speed stirring and dispersion can be carried out, and then the previous solution is poured into the vessel with very rapid stirring to induce adsorption and rapid cooling. At this step, the stirring is carried out at a very high speed for a time sufficient to enable very rapid adsorption and uniform dispersion of the solution.

As the excipient, there can be used at least one material selected from Tween 80 (polysorbate 80), PVP K-30 (polyvinylpyrrolidone), and talc (Mg₃(OH)₂Si₄O₁₀).

### MODE FOR THE INVENTION

Hereinafter, the pharmaceutical formulation of the present invention will be explained in more detail with reference to the following examples.

### EXAMPLES

### Example 1

10 g of polyethylene glycol 400, 10 g of polyoxyethylene castor oil (Cremophor), 10 g of polysorbate and 5 g of tocopherol acetate were heated to 40-60° C., and then 120 g of orlistat was added thereto. The mixture was homogeneously stirred to prepare a pale yellow transparent liquid formulation. The liquid state was transformed into an opaque coagulated state at room temperature.

Some portions were used to conduct a test for liquid stability. The other portions were adsorbed to an adsorbent, and then an excipient was added thereto. The resulting mixture was pressed to produce tablets, followed by coating with a film to obtain 800 tablet samples.

1) The liquid samples were cooled to form a coagulated material. The coagulated material had uniform shape and composition, and showed no phase separation and reaggregation. A series of storage at a low temperature of 4° C. and storage at a high temperature of 40° C. was repeated several times, and thereafter, a dissolution test was conducted. As a result, a high dissolution rate of 99.1% was obtained.

However, degradation of the liquid was observed after six weeks of storage under accelerated storage conditions. In addition, a 15% reduction in content was observed. Dark particles as degradation products and layers in which the dark particles were dispersed were observed.

2) The tablet samples were stored under accelerated conditions (temperature: 40° C., relative humidity: 70%).

Table 1 shows changes in dissolution rate and content of the tablet samples after six months of storage.

TABLE 1

| | | Change in |
|---|---|---|
| | Change in content | dissolution rate |
| Immediately after preparation | 102.3% | 98.6% |
| Six months after preparation | 98.5% | 98.5% |

### Example 2

10 g of polyoxyethylene castor oil (Cremophor) was heated to 40-60° C. to obtain a transparent liquid, and then 10 g of polysorbate was added thereto with gentle stirring. 120 g of orlistat was added to the mixture and homogeneously stirred to form a pale yellow transparent liquid formulation. The liquid state was transformed into an opaque coagulated state at room temperature.

Some portions were taken to observe the state of the liquid, and the other portions were adsorbed to produce tablets, followed by coating to obtain 800 tablet samples.

1) No phase separation and reaggregation were observed in the liquid samples. By the procedure of Example 1, a series of storage at a low temperature and storage at a high temperature was repeated several times, and thereafter, a dissolution test was conducted. As a result, a dissolution rate of about 59% was obtained. These observations indirectly show that the selected solvent in the present invention was suitable and inevitable to maximize the efficiency of the solubilizer, surfactant and antioxidant. After 2 weeks of storage under accelerated conditions, degradation products and layers thereof were observed.

2) The dissolution of the tablet samples was tested, and as a result, satisfactory results were not attained.

### Example 3

When 10 g of polyethylene glycol, 10 g of polysorbate and 5 g of tocopherol acetate were mixed with heating to obtain a transparent liquid, 120 g of orlistat was added to the mixture. The resulting mixture was homogeneously stirred to prepare a pale yellow transparent liquid formulation. The liquid state was transformed into an opaque semi-coagulated state at room temperature.

By the procedure of Example 1, some portions were separated, rapidly adsorbed, and pressed to produce tablets.

1) Phase separation, reaggregation and recrystallization of the separated liquid samples were observed during coagulation. By the procedure of Example 1, a series of storage at a low temperature and storage at a high temperature was repeated several times, and thereafter, a dissolution test was conducted. As a result, a dissolution rate of about 23% was obtained. These observations demonstrate that the solubilizer is an inevitable ingredient for stable dissolution of the formulation. After 2 weeks of storage under accelerated conditions, degradation products and layers thereof were observed.

2) The dissolution rate of the tablet samples was tested, and as a result, dissolution rates of the table samples were not significantly different from those of the liquid samples.

### Example 4

10 g of polyethylene glycol, 10 g of polyoxyethylene castor oil (Cremophor) and 5 g of tocopherol acetate were mixed and homogeneously stirred with heating to prepare a transparent solution, and then 120 g of orlistat was added to the solution. The mixture was homogeneously stirred to prepare a pale yellow transparent liquid formulation.

1) The liquid state was transformed into an opaque semi-coagulated state at room temperature. No phase separation and reaggregation were observed. A series of storage at a low temperature and storage at a high temperature was repeated several times, and thereafter, a dissolution test was conducted. As a result, a dissolution rate of 88% was obtained. However, a reduction in content was observed after four weeks of storage under accelerated storage conditions. Degradation products and layers thereof were observed.

2) An adsorbed powder was pressed to produce tablets, followed by coating to obtain tablet samples. The tablet samples were stored under accelerated conditions for 6 months. Table 2 shows changes in dissolution rate and content of the tablet samples during storage.

TABLE 2

| | | Change in |
|---|---|---|
| | Change in content | dissolution rate |
| Immediately after preparation | 101.5% | 92.0% |
| Six months after preparation | 97.5% | 88.4% |

The data shown in Table 2 demonstrate that the surfactant contributed significantly to dissolution of the pharmacologically active substance.

### Example 5

10 g of polyethylene glycol and 10 g of polyoxyethylene castor oil (Cremophor) were mixed and homogeneously stirred with heating to prepare a transparent solution, and then 120 g of orlistat was added to the solution. The mixture was homogeneously stirred to prepare a pale yellow transparent liquid formulation.

1) The liquid state was transformed into an opaque coagulated state at room temperature. No phase separation, recrystallization and reaggregation were observed. These observations demonstrate that the antioxidant was involved in recrystallization and reaggregation to prevent occurrence of phase separation during aggregation. A series of storage at a low temperature and storage at a high temperature was repeated several times, and thereafter, a dissolution test was conducted. As a result, a high dissolution rate of 95% was obtained. This suggests that phase separation and reaggregation during coagulation do not lead to a reduction in dissolution rate, and an optimum blending of the solvent, the solubilizer and the surfactant leads to effective dissolution. However, a reduction in the liquid content was observed after four weeks of storage under accelerated storage conditions. Degradation products and layers thereof were also observed.

2) An adsorbed powder was pressed to produce tablets, followed by coating to obtain coated tablet samples. The tablet samples were stored under accelerated conditions for 6 months. Table 3 shows changes in dissolution rate and content of the tablet samples during storage.

TABLE 3

| | | Change in |
|---|---|---|
| | Change in content | dissolution rate |
| Immediately after preparation | 102.4% | 97.6% |
| Six months after preparation | 78.5% | 95.3% |

As can be seen from the data shown in Table 3, tocopherol was significantly involved in the stability of the drug (i.e. orlistat).

Table 4 shows contents of the ingredients in each of the tablets produced in Examples 1 to 5.

TABLE 4

| | Exam- | Exam- | Exam- | Exam- | Exam- | Re- |
|---|---|---|---|---|---|---|
| Ingredients | ple 1 | ple 2 | ple 3 | ple 4 | ple 5 | marks |
| Orlistat | 120 | 120 | 120 | 120 | 120 | |
| Polyethylene | 10 | | 10 | 10 | 10 | |
| glycol 400 | | | | | | |
| Cremophor | 10 | 10 | | 10 | 10 | |
| Tween 80 | 10 | 10 | 10 | | | |
| Tocopherol | 5 | 5 | 5 | 5 | | |
| Microcrys- | 150 | 150 | 150 | 150 | 150 | |
| talline | | | | | | |
| cellulose | | | | | | |
| Silicon dioxide | 55.76 | 55.76 | 55.76 | 55.76 | | |
| Sodium starch | 30 | 30 | 30 | 30 | 30 | |
| glycolate | | | | | | |
| PVP K-30 | 12 | 12 | 12 | 12 | 12 | |
| Sodium lauryl | 7 | 7 | 7 | 7 | 7 | |
| sulfate | | | | | | |
| Talc | 0.24 | 0.24 | 0.24 | 0.24 | 0.24 | |
| Total | 410 | 400 | 400 | 400 | 405 | |

Surprisingly the present inventors have found that when a pharmacologically active substance is mixed with a solvent, a solubilizer agent and a surfactant for improving the solubility of the pharmacologically active substance to obtain an amorphous liquid or semi-solid state, an antioxidant is melted together with the mixture to solve poor chemical stability of the pharmacologically active substance in an amorphous or liquid state, and an adsorbent is strongly adsorbed to the molten mixture so as to be transformed into a powder form, the resulting very small molecules are reconstituted into crystal forms within the adsorbent to ensure chemical stability, the characteristic porous or cellulosic structure of the adsorbent blocks and protects the pharmacologically active substance from factors, e.g., air and moisture, causing chemical instability of the pharmacologically active substance, and the final pharmaceutical formulation is stabilized within a pH (as measured using an aqueous solution of 1 g of the pharmaceutical formulation in 100 ml of water) of 4.5 to 5.5. The present invention has been achieved based on these findings.

### INDUSTRIAL APPLICABILITY

As apparent from the above description, the pharmaceutical formulation of the present invention overcomes difficulties in the preparation of an active ingredient with a low melting point into a solid formulation, poor solubility of an active ingredient, and danger of chemical modifications during storage. In addition, according to the pharmaceutical formulation of the present invention, a drug can be stably dissolved despite changes in the environments of the body. Furthermore, since the pharmaceutical formulation of the present invention suitably takes advantage of low melting point and lipophilicity of a drug, it is economically advantageous. Moreover, the pharmaceutical formulation of the present invention has an advantage in that dangers of chemical changes resulting from high energy state of a liquid phase can be reliably avoided.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A pharmaceutical formulation with high stability and dissolution, the pharmaceutical formulation comprising 1 part by weight of orlistat, a lipase inhibitor, or its analogue as a pharmacologically active substance that is poorly soluble and has a low melting point, 0.01 to 20 parts by weight of a solvent, 0.01 to 20 parts by weight of a solubilizer, 0.01 to 10 parts by weight of a surfactant, 0.01 to 2 parts by weight of an antioxidant, and 0.1 to 20 parts by weight of an adsorbent or dispersant.

2. The pharmaceutical formulation according to claim 1, wherein the solvent is selected from almond oil, castor oil, corn oil, cotton seed oil, ethyl oleate, glycerin, glyceryl monostearate, olive oil, peanut oil, polyethylene glycol, propylene glycol, soy bean oil, and mixtures thereof.

3. The pharmaceutical formulation according to claim 1, wherein the solubilizer is selected from arabic gum, cetostearyl alcohol, cholesterol, diethanolamine, ethyl oleate, ethylene glycol palmitostearate, glycerin, glyceryl monostearate, hydroxypropyl cellulose, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, and mixtures thereof.

4. The pharmaceutical formulation according to claim 1, wherein the surfactant is selected from sodium docusate, glyceryl monooleate, polyethylene alkyl ether, polyoxyethylene sorbitan fatty acid ester (polysorbate=Tween), sodium lauryl sulfate, sorbic acid, sorbitan fatty acid ester, and mixtures thereof.

5. The pharmaceutical formulation according to claim 1, wherein the antioxidant is selected from tocopherol, ascorbic acid and its glycosides, butylated hydroxyanisole, citric acid, edetic acid, fumaric acid, malic acid, monothioglycerin, phosphoric acid, potassium metabisulfite, propionic acid, propyl gallate, tar acid, and mixtures thereof.

6. The pharmaceutical formulation according to claim 1, wherein the adsorbent or dispersant is selected from silicon dioxide, kaolin, magnesium aluminum silicate, cyclodextrin and its derivatives, alginic acid, propylene glycol alginate, gums, including arabic gum and xanthan gum, celluloses, including cellulose powder, microcrystalline cellulose, ethyl cellulose, methyl cellulose, calcium carboxymethyl cellulose, sodium carboxymethyl cellulose, hydroxyethyl cellulose, hydroxymethyl cellulose, hydroxypropyl cellulose and hydroxypropylmethyl cellulose, poloxamer, povidone and its derivatives, sodium starch glycolate, carbomer, dextrin, gelatin, medium-chain triglyceride, tragacanth, and mixtures thereof.

7. The pharmaceutical formulation according to claim 1, wherein the pharmaceutical formulation has a pH of 4.5 to 5.5.

8. A method for preparing a pharmaceutical formulation with high stability and dissolution, the method comprising the steps of:
mixing 0.01-20 parts by weight of a solvent, 0.01-20 parts by weight of a solubilizer, 0.01-10 parts by weight of a surfactant and 0.01-2 parts by weight of an antioxidant with heating to 40-60° C. (step S1);
mixing the mixture obtained in step S1 with 1 part by weight of a pharmacologically active substance (step S2);
adsorbing the mixture obtained in step S2 to 0.1-20 parts by weight of an adsorbent (step S3);
mixing the mixture obtained in step S3 with a pharmaceutically acceptable excipient suitable for molding (step S4); and
molding the mixture obtained in step S4 into a tablet followed by coating or capsule (step S5)
